(19) United States
(12) Patent Application Publication
(10) Pub. No.: US 2005/0090575 A1
(43) Pub. Date: Apr. 28, 2005

(54) Method and composition for making ceramic parts by stereolithophotography and use in dentistry

(75) Inventors: Christophe Chaput, Le Palais De Vienne (FR); Chartier Thierry, Feytiat (FR); Doreau Franck, Masleon (FR)

Correspondence Address: HELLER EHRMAN WHITE & MCAULIFFE LLP, 1717 RHODE ISLAND AVE, NQ, WASHINGTON, DC, 20036-3001, US

(73) Assignee: CENTRE DE TRANSFERT DE TECHNOLOGIES CERAMIQUES (C.T.T.C.), Limogas (FR)

(21) Appl. No.: 10/503,573

(22) PCT Filed: Feb. 7, 2003
(86) PCT No.: PCT/FR03/00391

(30) Foreign Application Priority Data
Feb. 8, 2002 (FR) 02/01599
Feb. 5, 2003 (FR) 03/01312

(51) Int. Cl. A61F 2/00

(52) U.S. Cl. 523/115

(57) ABSTRACT

The invention concerns a method wherein a pasty composition comprising a plasticizing agent is used in a selected amount to eliminate or reduce to a minimum stresses generated in the part during polymerization. The invention is useful in particular for making parts in dentistry.

The invention concerns making ceramic piece by stereolithography (rapid prototyping).

When making a ceramic piece by stereolithography, a thin layer of a composition containing a mixture of a ceramic powder, a photocurable resin, a photoinitiator, a dispersant and possible adjuvants is deposited on a support, this layer is cured in one or several selected zones by the action of suitable radiation, a new layer is deposited on the layer thus treated, and the operations are recommenced until all of the cured parts constitute the desired piece in the unprocessed state, the uncured piece are eliminated, the organic constituent of the unprocessed piece is eliminated, in particular by thermal decomposition (debinding), and the piece is sintered.

The method is implemented using a liquid or pasty composition.

In the case of a liquid composition, the support is immersed in a bath of the composition in such a way that it is covered only by said thin layer, and it is lowered gradually into the bath as the layers form.

A typical example is described in the publication U.S. Pat. No. 5,496,682 which indicates that the liquid must have a viscosity of less than 3 Pa·s, preferably less than 1 Pa·s.

In the case of a pasty composition, a suitable quantity of composition is deposited on the support in order to be spread across the latter by raking and form the desired layer, and the operation is repeated layer by layer.

A typical example is described in the publication FR 2 88 268.

Typically, the pasty composition has a viscosity of several hundred to several thousand Pa·s.

The piece obtained by stereolithography starting from a liquid composition are soft and have to undergo subsequent UV treatment (U.S. Pat. No. 5,496,682) in order to harden them and avoid their deformation during firing, whereas the piece obtained by stereolithography starting from a pasty composition have a total cure rate which induces a very rigid architecture since the grains cannot reorganize; the result of this is that there are very high stresses inside the piece during debinding, the polymer chains degrade, and the sudden release of these stresses can cause cracks during sintering, especially when the piece has a wall thickness of several millimeters.

The present invention aims to resolve this problem of cracking which arises in the case of stereolithography using a pasty solution.

In practice, these internal stresses existing in the raw piece are relaxed during debinding and generate cracking of the piece during sintering, especially when the piece has a wall thickness of several millimeters.

Surprisingly, it has been found that this problem could be resolved by incorporating a plasticizing agent into the paste, and that it was then possible to obtain, without any cracking, piece having a wall thickness of over 1 centimeter.

Incorporating a plasticizing agent into a composition for making a ceramic piece by rapid prototyping has already been proposed (publication U.S. Pat. No. 5,496,682), but this involved a method of stereolithography applied to a liquid composition, and the plasticizing agent had no function other than to reduce the viscosity of the composition so that the composition remains liquid.

This use could not therefore suggest using a plasticizing agent in a composition which is to remain pasty, and what is more to resolve a problem which only arises in the case of a pasty composition.

According to the present invention, the addition of plasticizing agent to the pasty composition is advantageously combined with the use of a quantity of ceramic powder sufficient to maintain the viscosity of the composition at a value of at least 10 000 Pa·s at a flow velocity gradient of 0.1 sec⁻¹ such that the paste is “self-holding”, that is to say does not flow by itself, and that the uncured parts of one layer can support the following layer.

According to the invention, the plasticizing agent eliminates or reduces to a minimum the internal stresses during photocuring and permits rapid relaxation of the possible residual stresses which may appear after exposure to radiation and are due to the curing kinetics, this by virtue of a reduction in the interactions between the chains, which favors their mobility.

The term “ceramic powder” designates one or more ceramic powders.

The word “resin” designates one or more resins.

In preferred embodiments, one or more of the following characteristics are implemented:
a pasty composition is used which comprises an alumina charge with a rate of at least 58% by volume of the volume of the composition;
a pasty composition is used which comprises an alumina charge with a rate of between 60 and 70% by volume of the volume of the composition;
a pasty composition is used which comprises an alumina charge with a rate of about 62-63% by volume of the volume of the composition;
a composition is used having an elastic modulus greater than the viscosity modulus;
a pasty composition is used which comprises a zirconia charge with a rate of 49 to 55% by volume of the volume of the composition;
a trifunctional photocurable resin is used;
an acrylate resin is used as photocurable resin;
an acrylate resin is used from the group formed by:
   di-ethoxylated bisphenol A dimethacrylate (for example Diacryl 101 from AKZO),
   1,6-hexanediol diacrylate (for example HDDA from UCB);
a pasty composition is used which comprises 20 to 50% by volume of plasticizing agent relative to the volume of the resin;
a pasty composition is used in which the plasticizing agent is one or more agents from the group formed by the family of glycols (e.g. polyethylene glycol), the family of phthalates (e.g. dibutylphthalate), glycerol.

Curing of acrylates is initiated through absorption of ultraviolet light by substances generating free radicals. The initiators of the acrylates are of the cationic type and their choice is guided principally by the wavelength of the light source they have to absorb, i.e. 350-360 mm in the case of ultraviolet.

Two particularly effective photoinitiators are the following:
2,2′-dimethoxy-2-phenylacetophenone (for example Irgacure 651 from CIBA)
2-hydroxy-2-methyl-1-phenyl-propan-1-one (for example Darocure 1173 from CIBA).

The dispersant used must be compatible with the photocurable resin in which it is dissolved and must be effective with the ceramic powder to be dispersed. The polyelectrolytes used in other ceramic processes are unsuitable because they do not easily dissociate in this type of medium. Dispersants with steric or electrosteric stabilization mechanisms are preferred.

Phosphoric esters have proven to be good dispersants.

Any plasticizing agent compatible with resins may be envisaged, in particular polyethylene glycol and glycerol in the case of acrylate resins. Dibutyl phthalate proves less effective.

### EXAMPLES

Pastes are prepared comprising (% by volume of the total volume):

| | Paste A | Paste B |
|---|---|---|
| Ceramic (1) | 62 | 59 |
| Resin (2) | 29.6 | 28.4 |
| Photoinitiator (3) | 0.1 | 1 |
| Dispersant (4) | 4.7 | 4.5 |
| Plasticizer (5) | 6.3 | 7.1 |

The viscosity of the paste at 0.1 sec⁻¹ is 14 200 Pa·s (paste A) and 13 200 Pa·s (paste B).
(1) alumina in paste A and hydroxyapatite oxyhapatite in paste B,
(2) Diacryl 101 from AKZO (paste A) and CN 503 from CRAY VALLEY (paste B),
(3) Irgacure 651 from CIBA,
(4) Beycostat A 259 from CECA (paste A) and C213 (paste B),
(5) PEG 300 from Merck.

Using paste A, rapid prototyping is performed with 188 layers of 100 microns to make a grille-shaped piece with overall dimensions of 230×230×13.8 mm, this piece being subjected to thermal treatment (debinding) up to 600° C., with a holding time of 2 hours at 600° C., then to sintering up to 1700° C., with a holding time of 1 hour 30 minutes at 1700° C.

The piece obtained has a flexural strength of 396 MPa.

Using paste B, rapid prototyping is performed with 230 layers of 100 microns to make a piece with overall dimensions of 72×37×23 mm, this part being subjected to thermal treatment (debinding) up to 600° C., with a holding time of 2 hours at 600° C., then to sintering up to 1400° C., with a holding time of 1 hour 30 minutes at 1400° C.

The piece obtained has a flexural strength of 102 MPa.

By way of comparison, a ceramic piece was produced by the bath technique using a liquid composition and where the ceramic charge rate is only 46.4% by volume:

| | |
|---|---|
| ceramic: | 151 cm³ |
| resin: | 91 cm³ |
| photoinitiator: | 5 cm³ |
| dispersant: | 39 cm³ |
| wetting agent: | 7.2 cm³ |
| plasticizer: | 32 cm³ |

Despite the low ceramic charge rate, preparation is difficult and necessitates the use of a solvent. The curing rate is very low. Production of a simple piece using this composition is possible only if it is of small size and without geometric detail (small rod, cube, cylinder). The unprocessed piece is very soft and deforms easily. Appearance of some delamination. After debinding and sintering, a small rod measuring 0.5×0.5×2 cm is completely fissured.

Among the possible applications of the invention, particular mention ought to be made of the application in dentistry.

Most dental bridges presently consist of a metal cap, which may or may not be covered with a porcelain. The latter, consisting of different ceramic layers which have been successively fired at high temperature, makes it possible to give the desired shade to the tooth or to all the teeth (bridge) to be implanted, so as to permit perfect integration thereof with the patient's dentition.

The dental structures are subjected to high mechanical stresses during their use, and the metal part is able to satisfy these demands. The main disadvantage, however, lies in the fact that several ceramic layers are necessary for:
masking the metal of the cap and giving good translucence and coloring;
ensuring the coefficients of dilation with the aim of obtaining a stable system free from microfissures.

The longevity of such systems depends on the quality of the bond between the metal and the first ceramic layer. The interface between these two materials is the source of defects, for example fissures.

With the aim of increasing the longevity of dental structures, of making coloration of the teeth easier, while at the same time limiting the number of layers generally of porcelain, the metal ring can be replaced by a ceramic ring.

The method and the composition of the present invention make it possible to obtain ceramic dental piece of small dimension, but with very precise dimensions adapted to each patient.

In this application, use will preferably be made of a stabilized zirconia powder, for example a zirconia stabilized with 3 mol % yttria (Y₃O₂). This stabilization makes it possible to retain a tetragonal microstructure and avoid any phase change causing fissuring of dense parts.

The stabilized zirconia has good mechanical properties (1200 MPa in 3 point flexion (supplier's data)), especially when the grains constituting the powder are very fine (<0.5 μm).

Production of the ceramic paste must be mastered in order to control the phenomena of rheology and reactivity. The use of fine zirconia powder allows the suspension to be charged with levels of between 49 and 55%. The viscosity at a flow velocity gradient of 0.1 is of the order of 10 000 to 15 000 Pa·s.

The production of dental structures requires the formation of ceramic piece having good tolerance properties. To do this, fine layers are formed during production. Their thickness is 25 um, making it posible to obtain a good surface state and greater precision. These low thicknesses are necessary for retaining reasonable curing speeds. This is because the low reactivity of zirconia-based pastes is a limiting factor with regard to the quantity of the pieces to be produced. The decrease in thickness of the layers makes it possible to reduce the curing time.

Working with such small thicknesses imposes more constraints during layer formation, and defects may be generated, such as local tears, lack of ceramic paste in places, etc. Organic products such as rheology agents make it possible to spread the paste in small thicknesses and obtain a correct layering. It is also possible to improve the surface state of each layer by addition of wetting agents or antifoaming agents, which products have the particularity of degassing the paste and of enhancing its spread during layering. These compounds make it possible to eliminate the defects at each layer.

The reactivity of the ceramic paste is an important parameter. In addition to the resin, it is possible to influence this characteristic by using a suitable photoinitiator or a mixture of photoinitiators. The aim of this is to be able to cure sufficient thicknesses to produce the desired part.

The composition advantageously comprises rheology agents so that the layers have surface states without any defects susceptible of creating microporosities.

An example of a ceramic paste is given below (% by volume of the total volume):

| | |
|---|---|
| ceramic powder: | ZrO₂ (49%) |
| resin: | CN5O3 from CRAY VALLEY (30.6%) |
| photoinitiators: | Irgacure 369 from CIBA |
| | Irgacure 819 from CIBA |
| | Irgacure 907 from CIBA |
| dispersant: | Beycostat C213 from CECA (5.5%) |
| plasticizer: | Dibutylphthalate from Acros |
| | Organics (4.6%) |
| rheology agents: | Rad 2100 from Tego (2.8%) |
| | Rad 2500 from Tego (2.9%) |
| | Glide 450 from Tego (2.9%) |
| antifoaming agent: | Foamex N from Tego (2.9%) |

Rapid prototyping technology adapted to dental structures permits tailor-made production of a cap or a bridge needed for a given patient. These structures can be simple individual caps or an assembly of caps (bridges) whose shape can be straight or curved.

A scanned image of the part to be repaired is processed using suitable software (CAO) making it possible to redimension the ceramic piece to be produced and cut it into successive sections of 25 μm.

The piece is then constructed physically by rapid prototyping with the paste described above.

Using this paste, rapid prototyping is performed to produce bridges measuring 40×6×12 mm³ and, after the uncured paste has been cleaned, these are subjected to thermal treatment (debinding) up to 550° C., with a holding time of 2 hours, then to sintering up to 1400° C., or even 1550° C., with a holding time of 2 hours, depending on the zirconia used.

The piece obtained has a flexural strength of the order of 1000 MPa.

In other embodiments, the zirconia is doped with alumina to increase the mechanical properties.

The invention is not limited to these examples.

1. A method for making a ceramic piece by a rapid prototyping procedure, which comprises operations in which a thin layer of a pasty composition containing a mixture of a ceramic powder, a photocurable resin, a photoinitiator, a dispersant and possible adjuvants is deposited on a support, this layer is cured locally in one or several selected zones by the action of suitable radiation, a new layer is deposited on the layer thus treated, and the operations are recommenced until all of the cured parts constitute the desired piece in the unprocessed state, the uncured parts are eliminated, the organic constituent of the unprocessed piece is eliminated by thermal decomposition (debinding), and this piece is sintered, characterized in that a plasticizing agent is incorporated into the composition in a selected quantity relative to the quantity of curable resin in order to eliminate or reduce to a minimum the stresses generated in the piece during curing so as to avoid cracking of the piece during sintering, and in that a composition is used containing a ceramic powder in a sufficient quantity to ensure that the composition has a viscosity of at least 10 000 Pa·s at a flow velocity gradient of 0.1 sec⁻¹, such that the paste is “self-holding”, that is to say does not flow by itself, and that the uncured parts of one layer can support the following layer.

2. A pasty composition for making a ceramic piece using the method as claimed in claim 1, which composition comprises a ceramic powder, a photocurable resin, a photoinitiator, a dispersant and possible adjuvants, characterized in that the composition comprises a plasticizing agent in a selected quantity to eliminate or reduce to a minimum the stresses generated in the piece during curing, so as to avoid cracking of the piece during sintering, and in that the proportion of ceramic powder is sufficient to ensure that the composition has a viscosity of at least 10 000 Pa·s at a flow velocity gradient of 0.1 sec⁻¹, such that the paste is “self-holding”, that is to say does not flow by itself, and that the uncured parts of one layer can support the following layer.

3. The composition as claimed in claim 2, which has an elastic modulus greater than the viscosity modulus.

4. The composition as claimed in one of claims 2 and 3, which comprises from 20 to 50% by volume of plasticizing agent relative to the volume of the resin.

5. The composition as claimed in one of claims 2 through 4, in which the plasticizing agent is one or more agents from the group formed by the family of glycols, the family of phthalates, and glycerol.

6. The composition as claimed in one of claims 2 through 5, which comprises, as photocurable resin, a trifunctional resin.

7. The composition as claimed in one of claims 1 through 6, and which comprises, as photocurable resin, an acrylate resin.

8. The composition as claimed in claim 7, in which the photocurable resin is a resin from the group formed by the following resins:
di-ethoxylated bisphenol A dimethacrylate
1,6-hexanediol diacrylate.

9. The composition as claimed in one of claims 2 through 8, which comprises at least 58% (by volume) of ceramic powder relative to the total volume of the composition.

10. The composition as claimed in claim 9, which comprises from 60 to 70% (by volume) of ceramic powder.

11. The composition as claimed in claim 10, which comprises about 62-63% (by volume) of ceramic powder.

12. The composition as claimed in one of claims 9 through 11, in which the ceramic powder is an alumina powder.

13. The composition as claimed in one of claims 2 through 8, in which the ceramic powder is a stabilized zirconia powder.

14. The composition as claimed in claim 13, and which contains 49 to 55% by volume of zirconia powder relative to the total volume of the composition.

15. The composition as claimed in claim 14, in which the zirconia is doped with alumina.

16. Use of the method as claimed in claim 1 and of the composition as claimed in one of claims 2 through 15 for producing caps, bridges and other dental structures.

17. Use as claimed in claim 16, in which the method is implemented using layers with a thickness of not more than 25 micrometers.
